(19) United States
(12) Patent Application Publication
(10) Pub. No.: US 2019/0091729 A1
(43) Pub. Date: Mar. 28, 2019

(54) Method and Apparatus for the Alloy-Dependent Sorting of Scrap Metal, in Particular Aluminum Scrap

(71) Applicants: Ronald Gillner, Swisttal (DE); Nils Robert Bauerschlag, Aachen (DE)

(72) Inventors: Ronald Gillner, Swisttal (DE); Nils Robert Bauerschlag, Aachen (DE)

(73) Assignee: Hydro Aluminium Rolled Products GmbH, Grevenbroich (DE)

(21) Appl. No.: 16/185,775

(22) Filed: Nov. 9, 2018

Related U.S. Application Data
(63) Continuation of application No. PCT/EP2017/061149, filed on May 10, 2017.

(30) Foreign Application Priority Data
May 11, 2016 (DE) 10 2016 108 745.9

(51) Int. Cl. B07C 5/342; G01N 33/202; G01N 21/71; G01N 23/223

(52) U.S. Cl. CPC B07C 5/3427; G01N 33/202; G01N 21/718; G01N 2223/076; B07C 2501/0054; B07C 2501/0036; G01N 2201/06113; G01N 23/223

(57) ABSTRACT

Disclosed is a method for sorting of scrap metal in which a composition analysis is carried out on a scrap fragment. Surface composition information about the local composition of the scrap fragment is determined, and associated volumetric composition information about the composition of the scrap fragment is assigned to the scrap fragment depending on the surface composition information determined by measurement and on a given assignment rule. Also disclosed is an apparatus for sorting scrap metal having a conveyor designed to convey a quantity of scrap fragments, an analysis device designed to carry out composition analyses on scrap fragments, and a control device designed to assign associated volumetric composition information about the composition of the scrap fragment. A composition analysis of a scrap fragment includes determining surface composition information about the local composition of the scrap fragment by measurement.

### CROSS-REFERENCE TO RELATED PATENT APPLICATIONS

This patent application is a continuation of PCT/EP2017/061149, filed May 10, 2017, which claims priority to German Application No. 10 2016 108 745.9, filed May 11, 2016, the entire teachings and disclosure of which are incorporated herein by reference thereto.

### FIELD

The invention relates to a method and an apparatus for the alloy-dependent sorting of scrap metal, in particular aluminum scrap.

### BACKGROUND

In the prior art, the sorting or recycling of aluminum takes place through several method steps. These usually comprise the collection of various aluminum scrap, mechanical processing of the scrap and a subsequent metallurgical utilization of the scrap.

For resource-efficient recycling, the mechanical processing of the scrap should produce an aluminum scrap product that meets the qualitative requirements of the metallurgical utilization method. For this purpose, different processing steps are carried out in the prior art, which allow, however, only a limited sorting by grade or alloy composition of the scrap.

The mechanical processing is usually carried out by a single or multi-stage comminution of the scrap, followed by various sorting steps. The sorting steps may, for example, effect separation of iron and non-ferrous metals via magnetic separators, air separation, eddy current separation, sensor-based sorting, for example by means of X-ray transmission or fluorescence, induction, laser-induced breakdown spectroscopy (LIBS) or near-infrared analysis (NIR). The procedural combination of these sorting steps allows the sorting of the scrap into different aluminum grades, i.e. in particular as a function of their alloy composition.

In order to sort various aluminum alloys alloy-specifically, one or more alloy elements of the individual scrap fragments must be determined. For this purpose, systems for laser-induced breakdown spectroscopy (LIBS) or X-ray fluorescence (XRF) are typically used. The analysis results generated with these systems are compared with given alloy compositions and the respective scrap fragments assigned to the appropriate alloy composition. If, for example, a 5% Mg content is determined during the analysis of a scrap fragment, this scrap fragment is assigned to an Mg5 alloy.

However, it has been found that despite the analysis of the scrap fragments, only a moderately good alloy-specific sorting of aluminum scrap can be achieved.

Against this background, the object of the present invention is to provide a method and an apparatus for sorting metal scrap, in particular aluminum scrap, which allow better sorting of the scrap.

### BRIEF SUMMARY

This object is achieved, at least in part, by method for the alloy-dependent sorting of metal scrap, in particular aluminum scrap, in which a composition analysis is performed on a scrap fragment, wherein surface composition information about the local composition in a surface region of the scrap fragment is determined by measurement on the scrap fragment, and in which associated bulk composition information about the composition of the scrap fragment in the bulk is assigned to the scrap fragment as a function of the surface composition information determined by measurement and a predetermined assignment rule.

In the method, composition analysis is performed on a scrap fragment, in which surface composition information about the local composition of a surface region of the scrap fragment is determined by measurement on the scrap fragment.

The surface composition information, in particular, comprises values and/or value ranges for the content of alloy elements, for example one or more alloy elements of the group Mg, Mn, Si or Fe.

The surface composition information comprises information about the local composition in a surface region of the scrap fragment. In the present case, a surface region of the scrap fragment is understood to be a near-surface volume of the scrap fragment. Surface-sensitive measurement methods, such as LIBS or XRF, have a limited analysis depth and, therefore, analyze the composition of a material only in a near-surface volume between the surface of the scrap fragment and the analysis depth. The surface composition information thus corresponds only to composition information that may be measured with such a surface-sensitive measurement method.

In the prior art, the composition information determined by such an analysis method, for example, by LIBS or XRF, is used for comparison with predetermined alloy compositions in order to associate the scrap fragments with one of these alloy compositions.

Within the scope of the invention, however, it has been recognized that this procedure may lead to misinterpretation, since the analysis methods used provide analysis results which may deviate from the actual overall composition of the scrap fragment.

In fact, due to segregation and diffusion effects in the material of the scrap fragment, the near-surface composition may in some cases partly differ significantly from the actual composition of the entire scrap fragment in the bulk. In particular, the segregation and diffusion effects lead to an element-specific enrichment of individual alloy elements on the surface of the scrap fragment. To make matters worse, the segregation and diffusion effects are differently pronounced for individual elements.

For example, in low-alloyed aluminum alloys, where the Mg content is 0.5% by weight in the bulk, near-surface enrichment of the Mg occurs, so that the Mg content at the surface is a factor of 10 above the actual Mg content in the bulk.

The near-surface analysis of the composition of the scrap fragment then provides a result that does not match the actual composition of the scrap fragment and thus leads to misinterpretation. For example, because of the high Mg content measured at the surface, the low-alloyed Mg0.5 alloy would be erroneously assigned to a higher-alloyed Mg5 alloy and correspondingly incorrectly sorted.

In principle, the penetration depth may be increased by increasing the laser power in laser-assisted methods. However, from a certain penetration depth, no longer sufficient light from the crater generated by the laser beam penetrates in the scrap fragment surface for the LIBS analysis, so that the analysis depth is still limited. Furthermore, the crater created by the laser beam is typically wider at the scrap fragment surface than at depth. For example, the crater may be cone-shaped. As a result, the LIBS analysis is dominated by the near-surface signal, as most of the material vaporizes from this region.

On the basis of this knowledge, in the case of the method described here, associated bulk composition information about the composition of the scrap fragment in the bulk, is assigned to the analyzed scrap fragment as a function of the surface composition information determined by means of measurement and a predetermined assignment rule. Instead of assigning the scrap fragment to an alloy solely based on the measured result of the composition analysis, which leads to the previously described misinterpretations in the prior art, a surface composition information determined by measurement is assigned to the scrap fragment on the basis of the bulk composition information assigned by the assignment rule, which characterizes the actual composition of the scrap fragment in the bulk.

In particular, the surface composition information and the bulk composition information associated with this surface composition information, differ at least partially from each other.

For example, surface composition information indicating an Mg content of 5% may be assigned to bulk composition information having an Mg content of 0.5%. As a result, the segregation and diffusion effects in the scrap component, which lead to a near-surface increase in the Mg content, are taken into account in the assignment, so that the scrap fragment may be further utilized suitably, in particular sorted.

In this way, the reliability and efficiency in the sorting of metal scrap, in particular aluminum scrap may be improved. In particular, analysis results are achieved for the scrap fragments, in which element-specific segregation and diffusion effects are taken into account.

Depending on the composition of the input scrap and the number of measured alloy elements, alloy-specific evaluation or assignment of the individual scrap fragments may be carried out in this way. This may then be used, for example, as a sorting criterion to separate scrap fragments of different alloys from each other. Furthermore, it is possible to use the analysis results as a sorting criterion for individual elements, for example, to separate scrap fragments having a particularly high or particularly low content of a specific alloy element.

In particular, the bulk composition information comprises values and/or ranges of values for the content of alloy elements, for example one or more of the group of alloys Mg, Mn, Si or Fe.

The above object is at least further partly achieved according to the invention by an apparatus for the sorting of metal scrap, in particular aluminum scrap, preferably for carrying out the method described above with a conveyor configured to convey a quantity of scrap fragments, with an analysis device configured to perform composition analyses of scrap fragments conveyed on the conveyor, wherein composition analysis of a scrap fragment comprises determination of surface composition information about the local composition in a surface region of the scrap fragment by means of measurement, and with a control device, which is configured to respectively assign associated bulk composition information about the composition of the scrap fragment in the bulk to the scrap fragments analyzed by the analysis device, as a function of the surface composition information determined by measurement and a predetermined assignment rule.

The apparatus comprises a conveyor which is designed to convey a quantity of scrap fragments. The conveyor may be, for example, a conveyor belt. With the conveyor, the scrap fragments may be conveyed through the device, in particular from a material inlet to the analysis device, so that the scrap fragments input at the material inlet onto the conveyor may be conveyed to the analysis device and analyzed there.

The apparatus further comprises an analysis device, which is configured to carry out composition analysis of scrap fragments conveyed on the conveyor. If the conveyor device is, for example, a conveyor belt, the analysis device may, in particular, comprise an analyzer which is arranged above the conveyor belt in order to examine the scrap fragments conveyed on the conveyor belt.

The composition analysis method, which can be performed on the scrap fragment by the analysis device, comprises determining surface composition information about the local composition in a surface region of the scrap fragment by a measurement. Accordingly, the entire composition of the scrap fragment is not detected by the analysis device, but the composition of the scrap fragment on its surface. In other words, a superficial composition analysis of the scrap component, preferably with a predetermined depth of analysis, is performed with the analysis device.

The apparatus further comprises a control device. In particular, the control device may comprise a microprocessor and a memory associated therewith and which comprises instructions, the execution of which by the microprocessor leads to processing of data and/or control of the apparatus.

The control device is configured to respectively assign associated bulk composition information about the composition of the scrap fragment in the bulk to the scrap fragment analyzed by the analysis device, as a function of the surface composition information determined by measurement and a predetermined assignment rule. For example, a function may be stored in a memory of the control device, which function calculates values for the contents of alloy components for the bulk composition information, from values for the contents of alloy components from the surface composition information by using defined algorithms. The algorithms are defined, in particular, so that they compensate an increase or a decrease of alloy elements at the surface area resulting from segregation and diffusion effects. For example, the algorithm may comprise a division of an Mg content from the surface composition information by the factor by which the Mg content at the surface is increased in proportion to the bulk due to diffusion and segregation effects, thereby determining the Mg content of bulk composition information to be assigned.

Various embodiments of the apparatus and the method are described below, wherein the individual embodiments may be used respectively both for the apparatus and for the method. Furthermore, the individual embodiments may be combined with one another as desired.

In a first embodiment of the method, a quantity of scrap fragments is provided, and a composition analysis is respectively carried out on a plurality of scrap fragments from the quantity of scrap fragments, in which surface composition information about the local composition in a surface region of the respective scrap fragment is determined by means of measurement on the respective scrap fragment, and associated bulk composition information about the composition of the respective scrap fragment in the bulk is assigned to the respective scrap fragment as a function of the surface composition information determined by measurement and a predetermined assignment rule. In this way, a quantity of scrap fragments may be analyzed in a rapid and reliable manner with scrap fragment accuracy (i.e. single grain accuracy) resolution. In particular, a quantity of scrap may be reliably characterized in this way. For example, an exact composition of the quantity of scrap may be determined. A composition determined in this way may then be used, for example, for furnace charging programs or for controlling scrap deliveries. Furthermore, the scrap fragments may be sorted based on the assigned bulk composition information.

The quantity of scrap fragments may be provided, for example, via a conveyor belt via which the scrap fragments are conveyed to a location for performing the composition analysis. Preferably, the scrap fragments are separated prior to performing the composition analysis, so that the individual scrap fragments are spatially separated from one another, for example, by being conveyed successively on a conveyor belt. This facilitates the analysis of the individual scrap fragments and the assignment of the respective bulk composition information to the individual scrap fragments. Accordingly, the apparatus preferably has a separating device, which is configured to separate scrap fragments before their analysis.

In a further embodiment of the method, the scrap fragment is sorted as a function of the assigned bulk composition information. When a quantity of scrap fragments is provided, the scrap fragments from the quantity of scrap fragments are accordingly sorted as a function of the respectively assigned bulk composition information. In a corresponding embodiment of the device, this comprises a sorting device, which is configured to sort the scrap fragments as a function of the bulk composition information respectively assigned to the scrap fragments by the control device.

The bulk composition information of a scrap fragment determined in the method, which bulk composition information characterize the actual bulk composition of the scrap fragment considerably more reliably than the measured surface composition information, may thus be used directly in order to sort the scrap fragment in an alloy-specific manner. In particular, a quantity of scrap fragments containing scrap fragments with several alloys may be sorted in this way.

The sorting device may, in particular, have a material feed, via which the scrap fragments are conveyed to the sorting device, and a sorter, which assigns the scrap fragments supplied via the material supply as a function of the bulk composition information, respectively associated to the scrap fragments, with one of at least two material discharges, with which material discharges the sorted scrap fragments are discharged of the sorting device.

In a further embodiment of the method, associated bulk composition information is assigned to the scrap fragment as a function of the surface composition information determined by measurement and a predetermined assignment rule in that bulk composition information is selected from a plurality of predetermined pieces of bulk composition information as a function of the surface composition information determined by means of measurement and the predetermined assignment rule.

For example, an assignment rule may be stored in a memory of the control device, which assignment rule associates to a plurality of predetermined alloy regions for the surface composition information in each case an alloy region for the surface composition information. Specifically, the association between an alloy region for the surface composition information and an alloy region for the bulk composition information is respectively selected so that scrap fragments of an alloy in the alloy region for the bulk composition information, when analyzed with the analysis apparatus, show surface composition information in the respectively assigned alloy region for the surface composition information. For surface composition information determined by measurement, the particular predetermined alloy region for the bulk composition information may then be selected, which is assigned to the predetermined alloy region for the surface composition information in which the surface composition information determined by measurement falls.

For this purpose, the surface composition information determined by measurement is compared, in particular, with the individual alloy regions for the surface composition information. Accordingly, in another embodiment of the method, a predetermined piece of surface composition information is respectively assigned to the predetermined piece of bulk composition information and the selection of the piece of bulk composition information from the plurality of predetermined pieces of bulk composition information takes place by a comparison of the measured surface composition information with the predetermined piece of surface composition information.

The selected alloy region for the bulk composition information may then be assigned to the corresponding scrap fragment as bulk composition information.

The assignment rule or the alloy regions for the surface composition information, the alloy regions for the bulk composition information, and the respective assignments to each other may, for example, be applied prior to performing the method described herein, by means of a layered composition analysis of typical scrap fragments, for instance by means of glow discharge optical emission spectroscopy (GDOES).

The GDOES analysis provides depth-dependent composition of the scrap fragment which allows determination of which bulk compositions manifest themselves due to segregation and diffusion effects and in which surface compositions. The result of this analysis allows an associated bulk composition information to be assigned to a measured surface composition information. In particular, the results of this analysis may be used to define the assignment rule or alloy regions for the surface composition information, and alloy regions for the bulk composition information, and respective assignments to each other.

In this way, the results of a rather elaborate GDOES analysis may be used to interpret the results of a faster analysis of the scrap fragments in the method described here, in particular by means of LIBS or XRF. In particular, the segregation and diffusion effects of aluminum alloys may be determined with sufficient accuracy via GDOES. The analysis results, which represent the element contents at different surface depths, may then, through the definition of a corresponding assignment rule, represent the database for the evaluation of the measurement results of, for example, a LIBS system, which LIBS system is then used for the subsequent sorting.

By the assignment of superficial measurement results of a LIBS system (for sorting) to the results of a GDOES analysis achieved in this way, significantly more accurate elemental analyses of individual scrap fragments may be achieved, which enables accurate alloy sorting.

The assignment rule may also consider correlations between individual alloy elements and their specific segregation and diffusion effects to assign the surface composition information determined by measurement to an associated bulk composition information. In particular, the assignment rule may make an assignment as a function of a ratio of the contents of two alloy elements, for example, the ratio of the Mg content to the Mn content of an aluminum alloy. In this way, for example, the ratio of the contents determined by the LIBS system at the analysis depth determined through the laser of the LIBS system, may be used practically as a fingerprint to obtain the surface composition information of a particular alloy, in particular an analysis value from a GDOES measurement, and thus to assign bulk composition information about the composition of the scrap fragment in the bulk.

In another embodiment of the method, in the composition analysis, surface composition information about the local composition in a surface region of the scrap fragment is determined, wherein the surface region extends from the surface of the scrap fragment to a known or predetermined depth (depth of analysis), for example, to a depth in the range of 1-100 μm, especially 1-10 μm. When using LIBS, the depth may be adjusted, for example, by the laser power and/or the choice of a particular laser type. By setting the type of laser and/or the laser power, the depth of analysis may be fixed and therefore known. Accordingly, the assignment rule is preferably selected as a function of a previously known or predetermined analysis depth.

By choosing the type of laser, the laser power and/or the optics used for the laser, the footprint of the laser beam, i.e. the size of the spot of the laser beam on the scrap fragment surface, and/or the shape of the crater created by the laser beam in the scrap fragment surface may be predetermined. Accordingly, the assignment rule is preferably selected as a function of the previously known or predetermined footprint and/or a previously known or predetermined crater shape. For example, it is conceivable that a laser beam creates a cylindrical, conical or spherical crater in the scrap fragment surface. In the case of a cylindrical crater, the signals from different depths contribute approximately equally to the overall result of the LIBS measurement. For a cone-shaped crater that tapers in depth, the near-surface areas of the crater dominate the overall result of the LIBS measurement. By taking into account the crater shape, therefore, a better assignment of the bulk composition information may be achieved.

Since the analysis depth is previously known, a more reliable assignment of bulk composition information to the surface composition information determined by measurement may take place. If, for example, as described above, a GDOES analysis is carried out to define the assignment rule, which provides the depth-dependent composition of scrap fragments, the surface composition information determined in a LIBS measurement may be determined by averaging or integrating the depth-dependent composition from the surface to the previously known analysis depth. By a weighted averaging or integration, the crater shape may be taken into account. In this way, the measurement result of the LIBS measurement for a scrap fragment of known composition may be predicted and thus an appropriate assignment rule defined.

In a further embodiment of the method, the composition analysis comprises a spectroscopic analysis, in particular a laser-induced breakdown spectroscopy (LIBS) or an X-ray fluorescence analysis (XRF). In a corresponding embodiment of the device, the analysis device comprises a spectroscopic analysis device, in particular an analysis device for laser-induced plasma spectroscopy (LIBS) or X-ray fluorescence analysis (XRF). Surface composition information of a scrap element may be measured quickly through the spectroscopic analysis. Laser-induced plasma spectroscopy and X-ray fluorescence analysis have been found to be particularly suitable methods to rapidly and reliably measure surface composition information. Since both methods are surface-sensitive, i.e. can determine the composition only in the surface region of the scrap fragment, immediate sorting as a function of a LIBS or XRF measurement is problematic. By means of the method described here or the device described here, LIBS and XRF measurements may now be used for a reliable alloy assignment of scrap fragments.

In a further embodiment of the method, the surface composition information determined by measurement comprises values for the contents of at least two alloy components of the scrap fragment. For example, the surface composition information may include values for the contents of two or more of the alloy elements Mg, Mn, Si or Fe. It has been found that the assignment of bulk composition information to surface composition information is more reliable when analyzing more than one alloy component of the scrap fragment. On the other hand, a maximum number of four alloy components is typically sufficient to reliably assign surface composition information so that the surface composition information preferably includes values for the contents of a maximum of four alloy components of the scrap fragment. As a result, the evaluation of the measured values determined with the analysis device may be simplified and carried out in a shorter time.

In another embodiment of the method, a quantity of scrap fragments is alloy-dependently sorted by performing the composition analysis and sorting for the individual scrap fragments from the quantity. Accordingly, a plurality of scrap fragments of a quantity are alloy-dependently sorted as a function of the respective determined composition analysis.

In another embodiment, the scrap fragments are separated before composition analysis is performed on the scrap fragments. In a corresponding embodiment of the apparatus, this apparatus comprises a separating device, which is configured to separate scrap fragments before they are fed to the analysis device. Through the separation, the scrap fragments have a predetermined order, with which they are conveyed through the apparatus. The assignment of selected bulk composition information to respective scrap fragments may therefore be made simply by assigning an order corresponding to the order of the scrap fragments to the bulk composition information. To sort the scrap fragments, the control device may then control the sorting device in the predetermined order as a function of the bulk composition information. Since the order of the scrap fragments remains as unchanged as possible during transport through the device, in this way each scrap fragment may be sorted according to the bulk composition information respectively assigned to this scrap fragment.

In a further embodiment, the apparatus comprises a detection device which is designed to detect the position of scrap fragments conveyed on the conveyor, wherein the control device is configured to control the analysis device and/or the sorting device as a function of the detected position of a scrap fragment. The detection device may, for example, comprise a camera or a laser scanner in order to detect the position of scrap fragments on the conveyor.

By controlling the analysis device as a function of the detected location of the scrap fragment, an accurate composition analysis may be performed. For example, in this way, a laser beam used for the analysis may be precisely aimed at the scrap fragment.

Accurate sorting may be performed since the sorting device is controlled as a function of the detected position of the scrap fragment. For example, in pneumatic sorting, an air blast may be directed at the targeted scrap fragment to supply it with a specific partial flow.

In particular, the order in which the scrap fragments are conveyed through the apparatus may also be detected with the detection device. As described above, this facilitates the assignment of the bulk composition information and the sorting of the scrap fragments.

In a further embodiment of the apparatus, the control device is configured to control the implementation of the previously described method or an embodiment thereof. Preferably, the control device comprises a processor and associated memory containing instructions, the execution of which on the processor implements the method or an embodiment thereof as described above. In this way, substantially automatic operation of the device is possible, in which the scrap fragments supplied to the apparatus are sorted alloy-specifically.

### BRIEF DESCRIPTION OF THE DRAWING

Further advantages and features of the method and the apparatus will become apparent from the following description of embodiments, in which reference is made to the accompanying drawings, in which:

FIG. 1 shows an embodiment of the device according to the invention,

FIG. 2 shows the analysis device of the apparatus of FIG. 1,

FIG. 3 shows the assignment of a bulk composition information,

FIG. 4 shows examples of measured depth-dependent composition information, and

FIG. 5 shows an embodiment of the method according to the invention.

### DETAILED DESCRIPTION

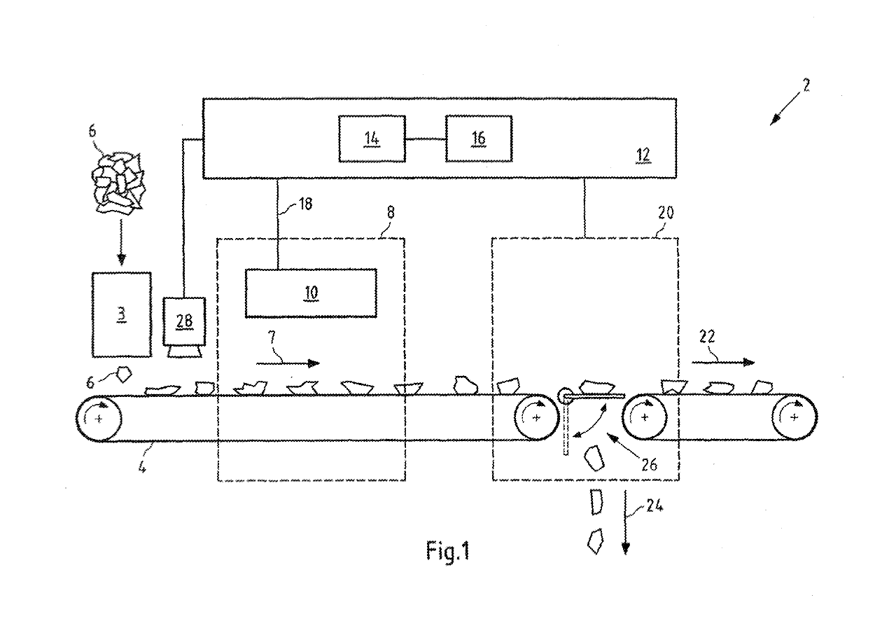

FIG. 1 shows an embodiment of the apparatus according to the invention for the sorting of metal scrap, in particular aluminum scrap, in a schematic representation. The apparatus 2 comprises a separating device 3 and a conveying device 4 in the form of a conveyor belt, with which scrap fragments 6 separated by the separating device 3 may be conveyed through the apparatus 2. The conveyance of the scrap fragments 6 through the apparatus 2 is represented in FIG. 1 by the material flow 7 indicated by an arrow.

Furthermore, the apparatus 2 has an analysis device 8 which is configured to carry out composition analysis on the scrap fragments 6 conveyed on the conveyor 4. For this purpose, the analysis device 8 comprises a spectroscopic analyzer 10, which may be, for example, an analyzer for laser-induced breakdown spectroscopy.

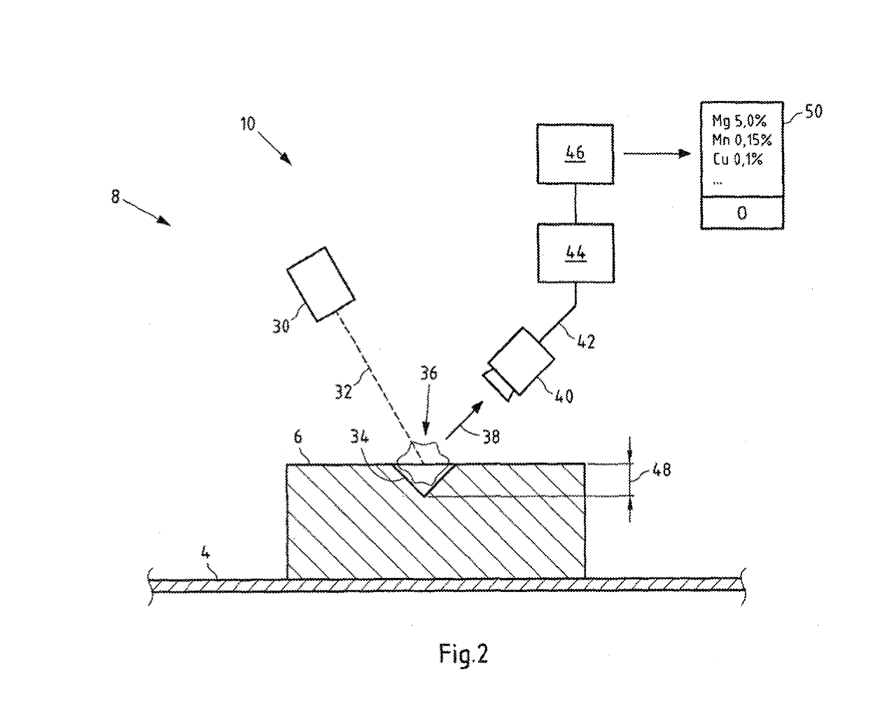

With the analyzer 10, surface composition information about the local composition in a surface region of the analyzed scrap fragment 6 may be determined by means of a measurement. Such an analysis will be explained in more detail below in connection with FIG. 2.

Furthermore, the apparatus 2 has a control device 12, which is configured to control the apparatus 2. For this purpose, the control device 12 comprises, in particular, a microprocessor 14 and a memory 16 connected thereto, which contains instructions the execution of which in the processor effects the control of the apparatus 2.

The analysis device 10 is connected to the control device 12 via a data connection 18 in order to transmit the surface composition information of a scrap fragment 6 determined by the analyzer 10 to the control device 12.

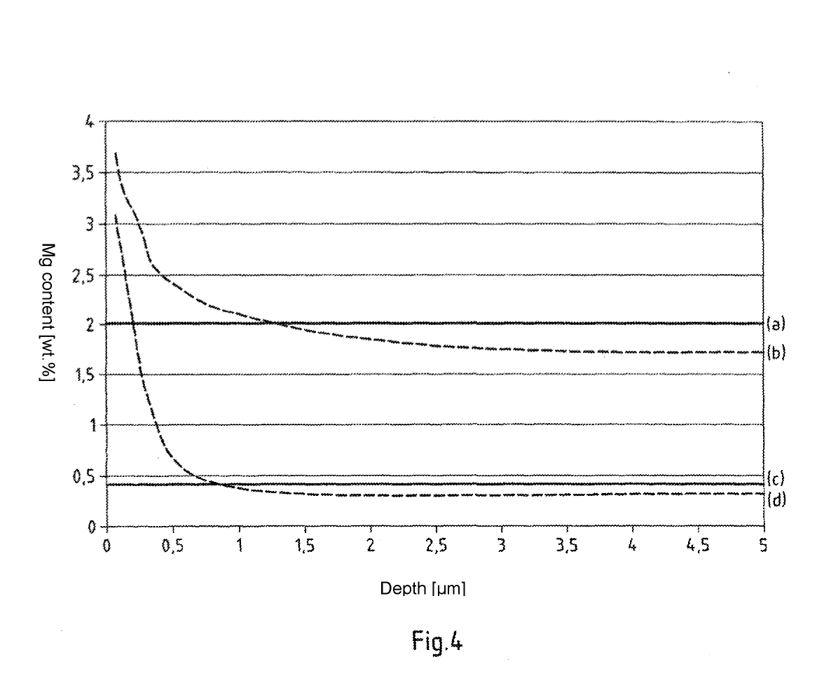

Furthermore, the control device 12 is designed to select bulk composition information associated with the surface composition information, as a function of the surface composition information received via the data connection 18 and a predetermined assignment rule stored in the memory 16, and to assign the bulk composition information to the scrap fragment analyzed by the analyzer 10. The selection of the bulk composition information is explained in more detail below in connection with FIG. 4. The assignment of the bulk composition information to the respectively associated scrap fragments 6 may be effected, for example, by assigning an order corresponding to the order of the scrap fragments 6 to the bulk composition information. Since the scrap fragments 6 are separated by the separator 3 and are therefore conveyed successively in a specific order by the apparatus 2, unambiguous assignment may be achieved in this way.

The apparatus 2 also has a sorting device 20, which is configured to sort the scrap fragments 6 as a function of the respectively assigned bulk composition information. The sorting of the scrap fragments 6 is shown schematically in FIG. 1 by the division of the material flow 7 into two partial flows 22 and 24. If the material flow 7 contains, for example, scrap fragments of low-alloyed aluminum alloys with an Mg and/or Mn content of max. 0.5 wt.-% and high-alloyed aluminum alloys, for example, with a Mg and/or Mn content of more than 2 wt.-%, then sorting of the scrap fragments 6 from the material flow 7 may be effected by the sorting device 20, in which scrap fragments, whose associated bulk composition information indicates a low Mg and Mn content, are assigned to the first partial flow 22, and the remaining high-alloyed scrap fragments are assigned to the second partial flow 24.

In order to assign a scrap fragment 6 to one of the partial flows 22, 24, the sorting device 20 shown in FIG. 1 has a controllable flap 26 which may be moved between a closed position (continuous line) and an open position (dashed line). When the flap 26 is in the closed position, a scrap fragment 6 conveyed on the conveyor belt 4 is assigned to the partial flow 22. When the flap 26 is in the open position, however, the scrap fragment 6 passes to the second partial flow 24. The controllable flap 26 is just one simple example of a way to divide the material flow 7 selectively into the partial flows 22, 24. Other means for sorting the scrap fragments 6 may be provided instead. For example, the individual scrap fragments may also be assigned pneumatically to one of the two partial flows 22, 24 by being conveyed into the respective partial flow by a short, strong burst of air.

Furthermore, the apparatus 2 also has a detection apparatus 28 in the form of a camera, with which the position of scrap fragments 6 conveyed on the conveyor 4 may be detected. For example, a laser scanner may be used instead of a camera.

The operation of the apparatus 2 will now be described.

A quantity of scrap fragments 6 is introduced into the separating device 3 and separated there, so that the scrap fragments 6 get successively on the conveyor 4 in a fixed order and are transported with this order through the apparatus.

The separated scrap fragments 6 are successively detected by the detection apparatus 28, by what the order of the scrap fragments 6 is detected.

The individual scrap fragments are then analyzed in the analysis device 8, and the control device 12 selects associated bulk composition information on the basis of the surface composition information determined during the analysis and of the predetermined assignment rule, and assigns these to the respective scrap fragment 6. The assignment of the bulk composition information is carried out in the present case by assigning the detected sequence of the scrap fragments 6 to the bulk composition information.

In the sorting device 20, the scrap fragments 6 are then supplied as a function of the respectively assigned bulk composition information each to one of the two partial flows 22, 24. To this end and based on the sequence of scrap fragments 6 detected with the detection device 28, it is determined which scrap fragment 6 gets next to the flap 26 and the flap 26 is controlled as a function of the bulk composition information assigned to this scrap fragment 6, so that the scrap fragment is fed to the correct partial flow 22, 24.

In this way, effective separation of different aluminum alloys from the material flow 7 is possible. The separation based on the respective bulk composition information assigned to the scrap fragments avoids misinterpretations and thus permits clean sorting of the individual scrap fragments.

In FIG. 1, two partial flows 22, 24 are exemplified. Of course, the material flow 7 may also be divided into more than two partial flows.

The sorting device 20 is shown in FIG. 1 as a unit spatially separated from the analysis device 8. However, the sorting device 20 and the analysis device 8 may also overlap spatially. For example, the sorting of the scrap fragments 8 may take place immediately after the analysis.

FIG. 2 shows a schematic illustration of the analysis device 8 of the apparatus 2 from FIG. 1. The analyzer 10 in this embodiment is an analyzer for laser-induced breakdown spectroscopy. The analyzer 10 comprises a laser source 30, with which a scrap fragment 6 conveyed on the conveyor belt 4 may be acted upon by a pulsed laser beam 32. The incident laser beam 32 vaporizes a small volume 34 on the surface of the scrap fragment 6 and ionizes it into a plasma 36. This creates a corresponding crater in the scrap fragment surface. Upon the breakdown of the plasma 36, light 38 is emitted that is characteristic of the alloy elements contained in the bulk 34. The volume 34 or the crater corresponding to the volume have a conical shape in this exemplary embodiment. The cross-section of the crater or of the volume 34 thus decreases with depth, so that the light 38 is predominantly dominated by the uppermost layers of the volume 34.

The analyzer 10 has optics 40 to capture the light 38 and pass it via a light guide 42 to a spectrometer 44, with which the spectral distribution of the light 38 may be analyzed. An evaluation device 46 connected to the spectrometer then calculates the composition of the volume 34 from the measured spectral distribution. Since the laser beam 32 has only a certain penetration depth 48, which is, as a function of the adjusted laser power, typically in the range of 1 to 10 μm, the evaluation device 46 delivers surface composition information 50, i.e. composition information about a near-surface volume of the scrap fragment material. The surface composition information 50 is labeled with a “0” for “surface” in FIG. 2 and contains contents of various alloy elements (Mg, Mn, Cu, etc.) in weight %. For apparatus 2, this surface composition information 50 is sent to the control device 12 via the data link 18 for further processing.

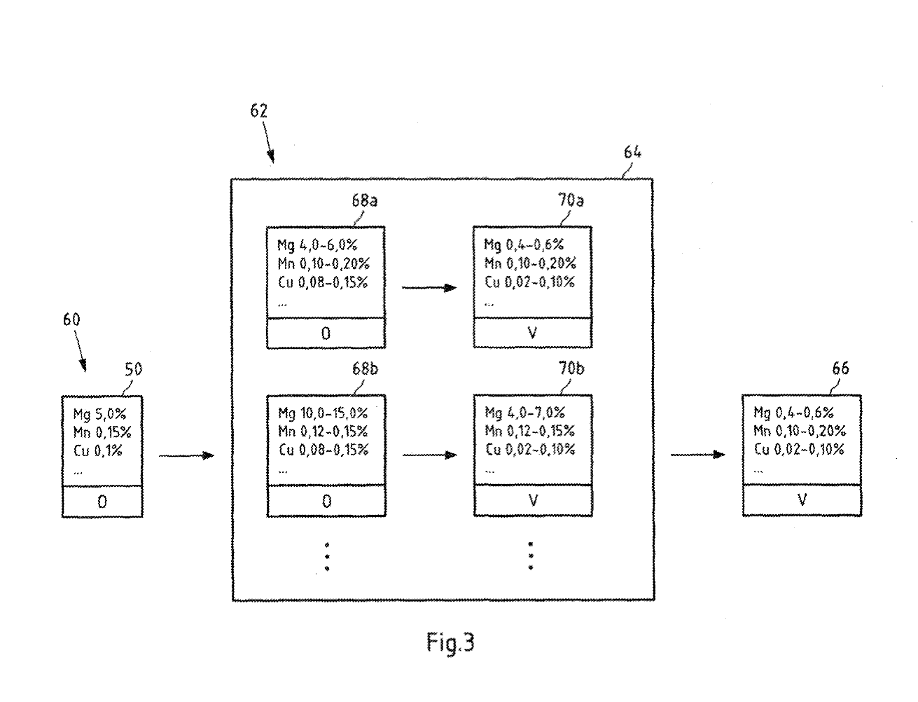

FIG. 3 schematically shows how the control device 12 assigns an associated bulk composition information to the surface composition information 50 measured by the analysis device 8.

First of all, the control device 12 receives in a first step 60 the surface composition information 50 measured by the analysis device 8.

In a second step 62, the control device 12 selects the associated bulk composition information 66 as a function of the surface composition information 50 and an assignment rule 64 stored in the memory 16. For this purpose, the assignment rule 64 in the present example comprises a table in which to a plurality of alloy regions for the surface composition information 68a, 68b, etc. an associated alloy region for the bulk composition information 70a, 70b is assigned in each case. The alloy regions shown numerically in the figure are exemplary.

The control device 12 compares the surface composition information 50 with the alloy regions for the surface composition information 68a, 68b and selects therefrom the appropriate alloy region for the surface composition information, which is the alloy region 68a in the present example. The alloy region for the bulk composition information 70a associated with this alloy region then represents the bulk composition information 66 assigned to the surface composition information 50, by which the sorting device 20 is controlled.

By selecting an associated bulk composition information 66 for the surface composition information 50 and controlling the sorting device 20 as a function of the bulk composition information 66 instead of the surface composition information 50, it is considered that the composition determined by the analysis device 8 on the surface of the scrap fragment 6 deviates from the actual bulk composition of the scrap fragment 6 due to segregation and diffusion effects. This allows a more reliable alloy-specific sorting of the scrap fragments based on the volumetric composition.

To determine the assignment rule, e.g. for a scrap fragment to be sorted, it is preferably examined which bulk compositions of a scrap fragment are reflected in which surface compositions. For example, individual sample fragments may be taken from a quantity of scrap fragments, such as a scrap quantity to be sorted, before being fed to the apparatus 2, for which individual sample fragments the surface composition, on the one hand, and the volumetric composition, on the other hand, are analyzed. The assignment rule 64 may then be defined from the relationships determined in this analysis between the surface composition and the bulk composition.

The analysis of the sample fragments may be investigated, for example, by glow discharge optical emission spectroscopy (GDOES). In this method, the sample fragment is used as a cathode in a DC plasma.

Through cathode sputtering, material is removed successively layer by layer from the surface of the sample fragment, wherein the removed atoms in the plasma emit characteristic light, which may be examined spectroscopically. In this way, the composition of the samples may be analyzed as a function of the depth.

FIG. 4 shows a diagram with measurement results of GDOES analysis on two scrap fragments. The diagram shows the depth-dependent composition of the scrap fragments using the example of the alloying element Mg. The abscissa indicates the depth in μm, i.e. the distance from the surface of the scrap fragment to the volume of the scrap fragment. The ordinate indicates the local Mg content in weight % at the corresponding depth.

The first analyzed scrap fragment consisted of an AA5XXX type aluminum alloy. The horizontal line (a) shows the average Mg content of the scrap fragment, which was determined by optical emission spectroscopy (OES). In the OES, the sparks penetrate deeper into the material and thus provide a value that corresponds to the average composition. Typically, a very accurate value for the bulk composition can be measured by an OES. The measurement curve (b) shows the depth-dependent Mg content of the scrap fragment determined by GDOES.

The second analyzed scrap fragment consisted of an AA6XXX type aluminum alloy. The horizontal line (c) in turn shows the average Mg content of the scrap fragment determined by optical emission spectroscopy (OES). The curve (d) shows the depth-dependent Mg content of the scrap fragment determined by GDOES.

It may be seen from the diagram that the Mg content directly at the surface of the scrap fragments (at 0-approx. 0.5 μm) is greatly increased by segregation and diffusion effects and is clearly above the respective average Mg content. At greater depth, the Mg content drops sharply and is even below the average Mg content in the investigated depth range of approx. 0.5 to 5 μm, since Mg has diffused from here to the surface.

The measurement results shown in FIG. 4 may be used, for example, to define a corresponding assignment rule 64, for example for a scrap delivery to be sorted. In particular, it may be deduced from the diagram which Mg content in the bulk for certain scrap fragments corresponds to a specific Mg content of a surface composition information.

By integrating or averaging the depth-dependent Mg content from curve (b) or (d) in the depth range from 0 μm to the penetration depth 48 of the laser beam 32, the Mg content may be determined, which Mg content results from the measurement with the analysis device 8 shown in FIG. 2 at the volume content along the line (a) or (c). The penetration depth 48 of the laser beam 32 depends essentially on the laser power and is thus known at a fixed laser power. For example, if the penetration depth 48 is set to 5 μm by adjusting the laser power, the Mg value of surface composition information may be calculated in particular by averaging the contents between 0 and 5 μm from the GDOES analysis. Preferably, the averaging or integration is carried out in a weighted manner in order to be able to take into account the footprint of the laser beam and/or the shape of the crater created by the laser beam in the scrap fragment surface. For example, in the cone-shaped crater illustrated in FIG. 2, near-surface contents are preferably weighted correspondingly more.

By means of these and corresponding analyses for further alloy elements, the alloy regions for the surface composition information 68a, 68b, etc., and the associated alloy regions for the bulk composition information 70a, 70b, etc. of the assignment rule 64 may thus be determined.

The diagram in FIG. 4 also shows that direct use of the Mg contents measured by the analysis device 8 to control the sorting device 20 without taking into account the assignment rule 64 would lead to considerable misinterpretations regarding the composition of the scrap fragments.

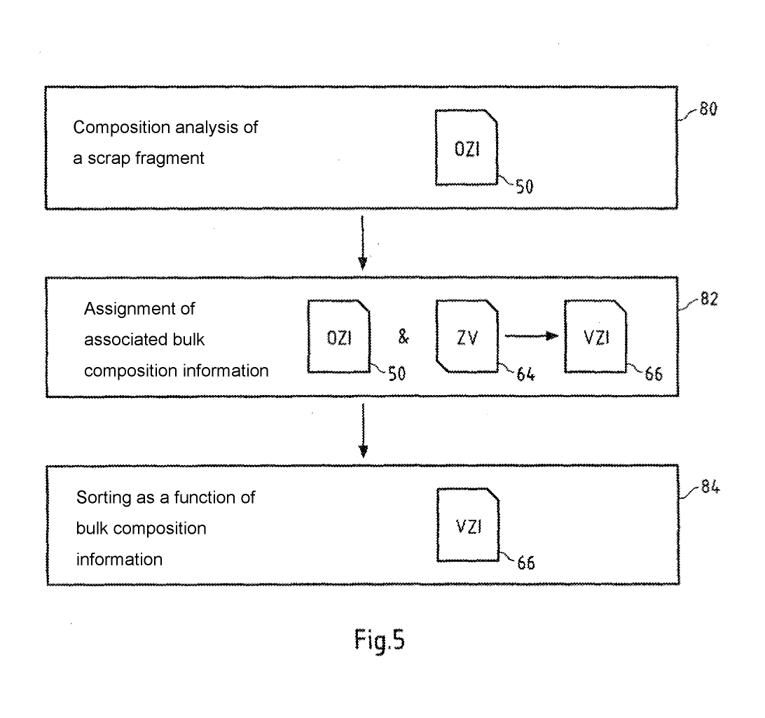

FIG. 5 shows an exemplary embodiment of a method according to the invention using the apparatus 2 shown in FIG. 1.

In a first step 80 of the method, scrap fragments 6 are supplied by the conveyor 4 to the analysis device 8 and there subjected to a composition analysis. For this purpose, the individual scrap fragments are subjected to a pulsed laser beam 32 by the analysis device 10, and respective surface composition information 50 is determined from the resulting light emission.

In a second step 82 of the method, the control device 12 selects an associated bulk composition information 66 for each surface composition information 50 by means of the assignment rule 64 and assigns this to the respective scrap fragment 6.

In a third step 84, the control device 12 controls the sorting device 20 so that the respective scrap fragment 6 is sorted as a function of the bulk composition information 66, i.e. in the present example it is assigned to one of the two partial flows 22 or 24.

As may be seen from the exemplary embodiments described above, an improved alloy-specific sorting of scrap fragments may be achieved with the described device and with the described method.

All references, including publications, patent applications, and patents cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) is to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method for the alloy-dependent sorting of scrap metal, in particular aluminum scrap, comprising:
performing a composition analysis on a scrap fragment, wherein surface composition information about the local composition in a surface region of the scrap fragment is determined by measurement on the scrap fragment; and
assigning associated bulk composition information about the composition of the scrap fragment in the bulk to the scrap fragment as a function of the surface composition information determined by measurement and a predetermined assignment rule.

2. The method according to claim 1, further comprising:
providing a quantity of scrap fragments;
respectively carrying out a composition analysis on a plurality of scrap fragments from the quantity of scrap fragments, wherein surface composition information about the local composition in a surface region of the respective scrap fragment is determined by means of measurement on the respective scrap fragment, and
assigning associated bulk composition information about the composition of the respective scrap fragment in the bulk to the respective scrap fragment as a function of the surface composition information determined by measurement and a predetermined assignment rule.

3. The method according to claim 1, further comprising:
sorting the scrap fragment as a function of the associated bulk composition information.

4. The method according to claim 1, further comprising:
assigning associated bulk composition information to the scrap fragment as a function of the surface composition information determined by measurement and a predetermined assignment rule in that bulk composition information is selected from a plurality of predetermined pieces of bulk composition information as a function of the surface composition information determined by means of measurement and the predetermined assignment rule.

5. The method according to claim 4, further comprising:
respectively assigning a predetermined piece of surface composition information to the predetermined piece of bulk composition information and the selection of the piece of bulk composition information from the plurality of predetermined pieces of bulk composition information takes place by a comparison of the measured surface composition information with the predetermined pieces of surface composition information.

6. The method according to claim 1, further comprising:
determining in the composition analysis, surface composition information about the local composition in a surface region of the scrap fragment, wherein the surface region extends from the surface of the scrap fragment to a known depth, in particular to a depth in the range of 2-10 μm.

7. The method according to claim 1, wherein the composition analysis comprises a spectroscopic analysis, in particular laser-induced breakdown spectroscopy (LIBS) or X-ray fluorescence analysis (XRF).

8. The method according to claim 1, wherein the surface composition information determined by measurement comprises values for the contents of at least two alloy components of the scrap fragment.

9. The method according to claim 2, further comprising:
separating the scrap fragments before a composition analysis is performed on the scrap fragments.

10. An apparatus for the sorting of metal scrap, in particular aluminum scrap, preferably for carrying out the method according to claim 1, comprising:
a conveyor configured to convey a quantity of scrap fragments;
an analysis device configured to perform composition analyses of scrap fragments conveyed on the conveyor, wherein composition analysis of a scrap fragment comprises determination of surface composition information about the local composition in a surface region of the scrap fragment by means of measurement; and
a control device which is configured to respectively assign associated bulk composition information about the composition of the scrap fragment in the bulk to the scrap fragments analyzed by the analysis device as a function of the surface composition information determined by measurement and a predetermined assignment rule.

11. The apparatus according to claim 10, further comprising a sorting device which is configured to sort scrap fragments as a function of the bulk composition information respectively assigned to the scrap fragments by the control device.

12. The apparatus according to claim 10, wherein the analysis device comprises a spectroscopic analysis device, in particular an analysis device for laser-induced breakdown spectroscopy (LIBS) or X-ray fluorescence analysis (XRF).

13. The apparatus according to claim 10, further comprising a separating device which is configured to separate scrap fragments before they are fed to the analysis device.

14. The apparatus according to claim 10, further comprising a detection device which is configured to detect the position of scrap fragments conveyed on the conveyor, wherein the control device is configured to control the analysis device and/or the sorting device as a function of the detected position of a scrap fragment.

15. The apparatus according to claim 10, wherein the control device is configured to control the implementation of:
performing a composition analysis on each scrap fragment, wherein surface composition information about the local composition in a surface region of each scrap fragment is determined by measurement on each scrap fragment; and
assigning associated bulk composition information about the composition of each scrap fragment in the bulk to each scrap fragment as a function of the surface composition information determined by measurement and a predetermined assignment rule.
